(19) United States
(12) Patent Application Publication
(10) Pub. No.: US 2005/0245507 A1
(43) Pub. Date: Nov. 3, 2005

(54) 7-Phenylsulfonyl-tetrahydro-3-benzazepine derivatives as antipsychotic agents

(76) Inventors: Ian Thomson Forbes, Harlow (GB); Andrew Derrick Gribble, Harlow (GB); Andrew Lightfoot, Harlow (GB); Andrew H. Payne, Harlow (GB); Graham Walker, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/516,065

(22) PCT Filed: May 28, 2003
(86) PCT No.: PCT/EP03/05728

§ 371 Date: Jun. 9, 2005

(30) Foreign Application Priority Data
May 29, 2002 (GB) 0212399.0

(51) Int. Cl. A61K 31/55; C07D 223/16

(52) U.S. Cl. 514/217.01; 540/594

(57) ABSTRACT

The invention provides compounds of formula (I): 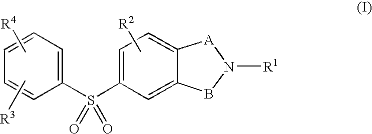 wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁵R⁶, —SO₂NR⁵R⁶, —(CH₂)ₚNR⁵R⁶, —(CH₂)ₚNR⁵COR⁶, optionally substituted aryl ring, optionally substituted heteroaryl ring or optionally substituted heterocyclyl ring;
R³ represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸ or —(CH₂)ₚNR⁷COR⁸;
R⁴ represents hydrogen, hydroxy, C₁₋₆alkyl, C₁₋₆alkoxy, C₁₋₆fluoroalkoxy, trifluoromethyl, trifluoromethoxy, halogen, —OSO₂CF₃, —(CH₂)ₚC₃₋₆cycloalkyl, —(CH₂)_{q}OC₁₋₆alkyl or —(CH₂)ₚOC₃₋₆cycloalkyl;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
R⁷ and R⁸ each independently represent hydrogen or C₁₋₆alkyl; m and n independently represent an integer selected from 1 and 2; p independently represents an integer selected from 0, 1, 2 and 3; q independently represents an integer selected from 1, 2 and 3; or a pharmaceutically acceptable salt or solvate thereof, with the proviso that the compounds 8-hydroxy-3-methyl-7-phenylsulfonyl-2,3,4,5-tetrahydro-1H-3-benzazepine, 8-hydroxy-7-4-(hydroxyphenyl)sulfonyl-2,3,4,5-tetrahydro-1H-3-benzazepine, 7-phenylsulfonyl-1,2,3,4-tetrahydroisoquinoline and 7-phenylsulfonyl-1,2,3,4-tetrahydroisoquinoline hydrochloride are excluded. The compounds are useful in therapy, in particular as antipsychotic agents.

This invention relates to novel compounds, pharmaceutical compositions containing them and their use in therapy, in particular as antipsychotic agents.

EP285287 describes 3-benzazepine compounds for use in treating gastrointestinal motility disorders including the compounds 8-hydroxy-3-methyl-7-phenylsulfonyl-2,3,4,5-tetrahydro-1H-3-benzazepine and 8-hydroxy-7-4-(hydroxyphenyl)sulfonyl-2.3,4,5-tetrahydro-1H-3benzazepine.

J. Med. Chem. 1999, 42, 118-134 and Biorg. Med. Chem. Lett, 1999, 9(3), 481-486 describe 7-substituted-1,2,3,4-tetrahydroisoquinolines and their relative affinities toward phenylethanolamine N-methyltransferase, including the compound 7-phenylsulfonyl-1,2,3,4-tetrahydroisoquinoline and its hydrochloride.

International patent applications WO98/06699, WO97/43262 and WO02/40471 (SmithKline Beecham) disclose tetrahydroisoquinoline and tetrahydrobenzazepine derivatives which are selective D3 receptor antagonists and are said to be useful as antipsychotic agents. All of these derivatives possess a group other than hydrogen or alkyl attached to the nitrogen atom of the tetrahydroisoquinoline or tetrahydrobenzazepine ring.

International patent application WO98/12180 (BASF) discloses hetaroyl cyclohexanedione derivatives including tetrahydroisoquinoline derivatives that are said to be useful for controlling harmful plants.

International patent application WO02/46164 (AstraZeneca) discloses tetrahydroisoquinoline and isoindoline derivatives that are ER-β-selective ligands and are said to be useful in the treatment or prophylaxis of Alzheimer's disease, anxiety disorders, depressive disorders, osteoporosis, cardiovascular disease, rheumatoid arthritis or prostate cancer.

International patent application WO 01/85695 discloses tetrahydroisoquinoline analogues useful as growth hormone secretagogues. Such analogues are also said to be useful in the treatment of disorders including inter alia, obesity, schizophrenia, depression and Alzheimer's disease.

Japanese patent application JP2001/19676 (Takeda) describes the synthesis of tetrahydrobenzazepine derivatives that are said to be useful for increasing cAMP concentration in mammals and, in particular, for treating obesity.

We have now found a novel group of phenylsulfonyl compounds which are useful particularly as antipsychotic agents.

According to the invention, there is provided a compound of formula (I): 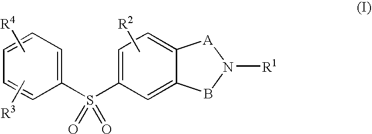
wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁵R⁶, —SO₂NR⁵R⁶, —(CH₂)ₚNR⁵R⁶, —(CH₂)ₚNR⁵COR⁶, optionally substituted aryl ring, optionally substituted heteroaryl ring or optionally substituted heterocyclyl ring;
R³ represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸ or —(CH₂)ₚNR⁷COR⁸;
R⁴ represents hydrogen, hydroxy, C₁₋₆alkyl, C₁₋₆alkoxy, C₁₋₆fluoroalkoxy, trifluoromethyl, trifluoromethoxy, halogen, —OSO₂CF₃, —(CH₂)ₚC₃₋₆cycloalkyl, —(CH₂)_{q}OC₁₋₆alkyl or —(CH₂)ₚOC₃₋₆cycloalkyl;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
R⁷ and R⁸ each independently represent hydrogen or C₁₋₆alkyl;
m and n independently represent an integer selected from 1 and 2;
p independently represents an integer selected from 0, 1, 2 and 3;
q independently represents an integer selected from 1, 2 and 3;
or a pharmaceutically acceptable salt or solvate thereof,
with the proviso that the compounds 8-hydroxy-3-methyl-7-phenylsulfonyl-2,3,4,5-tetrahydro-1H-3-benzazepine, 8-hydroxy-7-4-(hydroxyphenyl)sulfonyl-2,3,4,5-tetrahydro-1H-3-benzazepine, 7-phenylsulfonyl-1,2,3,4-tetrahydroisoquinoline and 7-phenylsulfonyl-1,2,3,4-tetrahydroisoquinoline hydrochloride are excluded.

It is to be understood that the present invention covers all combinations of particular and preferred groups described herein above.

As used herein, the term “alkyl” refers to straight or branched hydrocarbon chains containing the specified number of carbon atoms. For example, C₁₋₆alkyl means a straight or branched alkyl containing at least 1, and at most 6, carbon atoms. Examples of “alkyl” as used herein include, but are not limited to, methyl, ethyl, n-propyl, n-butyl, n-pentyl, n-hexyl, isobutyl, isopropyl, t-butyl and 1,1-dimethylpropyl.

As used herein, the term “alkoxy” refers to a straight or branched alkoxy group containing the specified number of carbon atoms. For example, C₁₋₆alkoxy means a straight or branched alkoxy group containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, 2,2,2-trifluoroethoxy, 2,2-dimethylprop-1-oxy, —O—CH₂-c-propyl, pentoxy or hexyloxy.

As used herein, the term “C₁₋₆fluoroalkoxy” refers to a straight or branched alkoxy group containing the specified number of carbon atoms wherein any of the carbon atoms may be substituted by one or more fluorine atoms. Examples of “C₁₋₆fluoroalkoxy” as used herein include, but are not limited to, 2,2,2-trifluoroethoxy.

As used herein, the term “cycloalkyl” refers to a non-aromatic hydrocarbon ring containing the specified number of carbon atoms. For example, C₃₋₇-cycloalkyl means a non-aromatic ring containing at least three, and at most seven, ring carbon atoms. Examples of “cycloalkyl” as used herein include, but are not limited to, cyclopropyl, cyclobutyl, cyclopentyl, cyclohexyl and cycloheptyl. A C₆₋₇cycloalkyl group is preferred.

As used herein, the term “halogen” refers to the elements fluorine, chlorine, bromine and iodine. Preferred halogens are fluorine, chlorine and bromine.

As used herein, the term “aryl” refers to a phenyl or a naphthyl ring.

As used herein, the term “heteroaryl” refers to a 5- or 6-membered heterocyclic aromatic ring or a fused bicyclic heteroaromatic ring system.

As used herein, the term “heterocyclyl” refers to a 3- to 7-membered monocyclic saturated ring containing at least one heteroatom independently selected from oxygen, nitrogen and sulfur. Examples of suitable heterocyclic rings include, but are not limited to, piperidine and morpholine.

As used herein, the term “5- or 6-membered heterocyclic aromatic ring” refers to a monocyclic unsaturated ring containing at least one heteroatom independently selected from oxygen, nitrogen and sulfur. Examples of suitable 5- and 6-membered heterocyclic aromatic rings include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl and isoxazolyl.

As used herein, the term “fused bicyclic heteroaromatic ring system” refers to a ring system comprising one six-membered unsaturated ring and one 5- or 6-membered unsaturated ring fused together, the ring system containing at least one heteroatom independently selected from oxygen, nitrogen and sulfur. Examples of suitable fused bicyclic heteroaromatic ring systems include, but are not limited to, indolyl, benzofuranyl, quinolyl and benzothienyl.

As used herein, the term “azacycloalkyl ring” refers to a 4- to 7-membered monocyclic saturated ring containing one nitrogen atom. Examples of suitable azacycloalkyl rings are azetidine, pyrrolidine, piperidine and azepine.

As used herein, the term “oxo-substituted azacycloalkyl ring” refers to an azacycloalkyl ring as defined above substituted by one oxo group. Examples of suitable oxo-substituted azacycloalkyl rings include, but are not limited to, azetidinone, pyrrolidinone, piperidinone and azepinone.

As used herein, the term “substituted” refers to substitution with the named substituent or substituents, multiple degrees of substitution being allowed unless otherwise stated.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include water, methanol, ethanol and acetic acid. Most preferably the solvent used is water and the solvate may also be referred to as a hydrate.

It will be appreciated that for use in medicine the salts of formula (I) should be physiologically acceptable. Suitable physiologically acceptable salts will be apparent to those skilled in the art and include for example acid addition salts formed with inorganic acids e.g. hydrochloric, hydrobromic, sulfuric, nitric or phosphoric acid; and organic acids e.g. succinic, maleic, malic, mandelic, acetic, fumaric, glutamic, lactic, citric, tartaric, benzoic, benzenesulfonic, p-toluenesulfonic, methanesulfonic or naphthalenesulfonic acid. Other non-physiologically acceptable salts e.g. oxalates, may be used, for example in the isolation of compounds of formula (I) and are included within the scope of this invention. Also included within the scope of the invention are solvates and hydrates of the compounds of formula (I).

Certain of the compounds of formula (I) may form acid addition salts with one or more equivalents of the acid. The present invention includes within its scope all possible stoichiometric and non-stoichiometric forms thereof.

Certain compounds of formula (I) may exist in stereoisomeric forms (e.g. they may contain one or more asymmetric carbon atoms). The individual stereoisomers (enantiomers and diastereomers) and mixtures of these are included within the scope of the present invention. The present invention also covers the individual isomers of the compounds represented by formula (I) as mixtures with isomers thereof in which one or more chiral centres are inverted. Likewise, it is understood that compounds of formula (I) may exist in tautomeric forms other than that shown in the formula and these are also included within the scope of the present invention.

The groups R², R³ and R⁴ may be located on any position on their respective phenyl rings. When R² represents an optionally substituted aryl ring, an optionally substituted heteroaryl ring, or an optionally substituted heterocycdyl ring, the optional substituents may be selected from C₁₋₆alkyl, C₁₋₆alkoxy, halogen, trifluoromethyl, trifluoromethoxy, cyano and —S—C₁₋₆alkyl.

Preferably, R¹ represents hydrogen or C₁₋₄alkyl. More preferably, R¹ represents hydrogen, methyl, ethyl, n-propyl or isopropyl. Even more preferably, R¹ represents hydrogen, methyl, ethyl or isopropyl.

Preferably, R² represents hydrogen, halogen, C₁₋₆alkyl or C₁₋₆alkoxy. More preferably, R² represents hydrogen, halogen, C₁₋₄alkyl or C₁₋₄alkoxy. Even more preferably, R² represents hydrogen, methoxy or bromo.

In a first embodiment of the invention, the R² group is located at the para-position relative to the group B i.e. a compound of formula (IA) 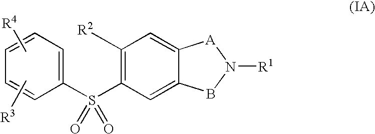
or a pharmaceutically acceptable salt or solvate thereof wherein groups A, B and R¹ to R⁴ have any of the meanings as given hereinbefore.

When R² is located in the para-positim i.e. compounds of formula (IA), R² is preferably hydrogen or methoxy.

For compounds of the formulae (I) or (IA), preferably, when R² represents an optionally substituted aryl ring, an optionally substituted heteroaryl ring, or an optionally substituted heterocyclyl ring, the optional substituents are independently selected from chlorine, fluorine, bromine, methyl, ethyl, t-butyl, methoxy, trifluoromethyl, trifluoromethoxy, cyano, —S-methyl and —NR⁵R⁶ wherein R⁵ and R⁶ are as hereinbefore described.

For compounds of the formulae (I) or (IA), preferably, R³ represents hydrogen, hydroxy, C₁₋₄alkyl or C₁₋₄alkoxy. More preferably, R³ represents hydrogen, methyl or methoxy. Even more preferably, R³ represents hydrogen, methyl or methoxy.

In another embodiment of the invention, the R³ group is located at the ortho-position relative to the sulfone group i.e. a compound of formula (IB) 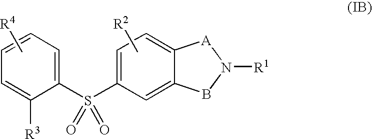
or a pharmaceutically acceptable salt or solvate thereof wherein groups A, B and R¹ to R⁴ have any of the meanings as given hereinbefore.

When R³ is located in the ortho-position i.e. compounds of formula (IB), R³ is preferably hydrogen, methyl or methoxy.

For compounds of the formulae (I), (IA) or (IB), preferably, R⁴ represents hydrogen, C₁₋₄alkyl, C₁₋₄alkoxy, C₁₋₄fluoroalkoxy, trifluoromethyl, trifluoromethoxy, halogen or —OSO₂CF₃. More preferably, R⁴ represents C₁₋₄alkyl or C₁₋₄alkoxy. More preferably, R⁴ represents isopropyl, n-butyl, t-butyl, ethoxy, propoxy, isopropoxy, trifluoromethoxy, —OSO₂CF₃, 2,2,2-trifluoroethoxy, 2,2-dimethylprop-1-oxy, —OCH₂-c-propyl, or pentoxy.

In another embodiment of the invention, the R⁴ group is located at the meta-position relative to the sulfone group i.e. a compound of formula (IC) 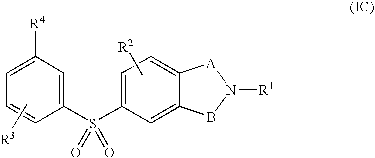
or a pharmaceutically acceptable salt or solvate thereof wherein groups A, B and R¹ to R⁴ have any of the meanings as given hereinbefore.

When R⁴ is located in the meta-position i.e. compounds of formula (IC), R⁴ is preferably C₁₋₄alkyl or C₁₋₄alkoxy.

In another embodiment of the invention, the R⁴ group is located at the para-position relative to the sulfone group i.e. a compound of formula (ID) 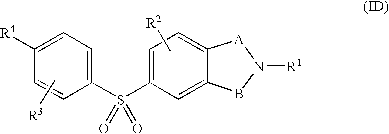
or a pharmaceutically acceptable salt or solvate thereof wherein groups A, B and R¹ to R⁴ have any of the meanings as given hereinbefore.

When R⁴ is located in the para-position i.e. compounds of formula (ID), R⁴ is preferably C₁₋₄alkyl or C₁₋₄alkoxy. More preferably, R⁴ represents isopropyl, n-butyl, t-butyl, ethoxy, propoxy, isopropoxy, trifluoromethoxy, —OSO₂CF₃, 2,2,2-trifluoroethoxy, 2,2-dimethylprop-1-oxy, —OCH₂-c-propyl, or pentoxy.

In another embodiment of the invention, the R³ group is located at the ortho-position relative to the sulfone group and the R⁴ group is located at the para-position relative to the sulfone group i.e. a compound of formula (IE) 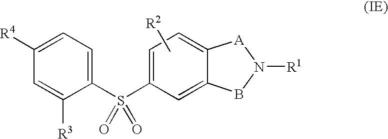
or a pharmaceutically acceptable salt or solvate thereof wherein groups A, B and R¹ to R⁴ have any of the meanings as given hereinbefore.

For compounds of the formulae (I), (IA), (IB), (IC), (ID) or (IE), preferably, R⁵ and R⁶ independently represent hydrogen or C₁₋₄alkyl. More preferably, R⁵ and R⁶ independently represent hydrogen or methyl.

For compounds of the formulae (I), (IA), (IB), (IC), (ID) or (IE), preferably, R⁷ and R⁸ independently represent hydrogen or C₁₋₄alkyl. More preferably, R⁷ and R⁸ independently represent hydrogen or methyl.

For compounds of the formula (I), (IA), (IB), (IC), (ID) or (IE), preferably, p represents 0. In another embodiment of the invention, m is 1 and n is 1 and the invention is a compound of formula (IF): 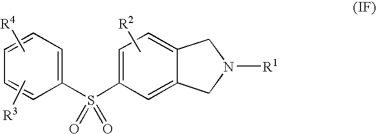
or a pharmaceutically acceptable salt or solvate thereof wherein groups R¹ to R⁴ have any of the meanings as given hereinbefore.

In another embodiment of the invention, m is 2 and n is 1 and the invention is a compound of formula (IG): 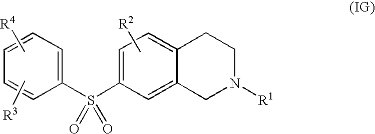
or a pharmaceutically acceptable salt or solvate thereof wherein groups R¹ to R⁴ have any of the meanings as given hereinbefore.

In another embodiment of the invention, m is 1 and n is 2 and the invention is a compound of formula (IH): 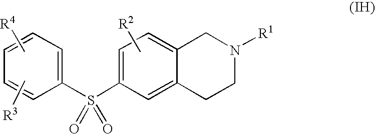
or a pharmaceutically acceptable salt or solvate thereof wherein groups R¹ to R⁴ have any of the meanings as given hereinbefore.

In another embodiment of the invention, m is 2 and n is 2 and the invention is a compound of formula (IJ): 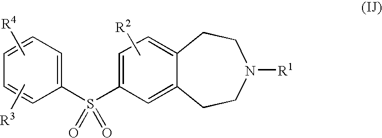
or a pharmaceutically acceptable salt or solvate thereof wherein groups R¹ to R⁴ have any of the meanings as given hereinbefore.

In another embodiment of the invention, m is 2 and n is 2, the R² group is located at the para-position relative to the group B, the R³ group is located at the ortho-position relative to the sulfone group, the R⁴ group is located at the meta-position relative to the sulfone group and the invention is a compound of formula (IK): 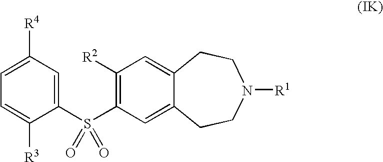
or a pharmaceutically acceptable salt or solvate thereof wherein groups R¹ to R⁴ have any of the meanings as given hereinbefore.

In another embodiment of the invention, m is 2 and n is 2, the R² group is located at the para-position relative to the group B, the R³ group is located at the ortho-position relative to the sulfone group, the R⁴ group is located at the para-position relative to the sulfone group and the invention is a compound of formula (IL): 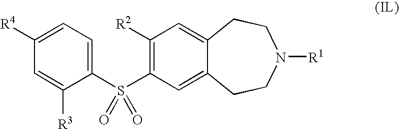
or a pharmaceutically acceptable salt or solvate thereof wherein groups R¹ to R⁴ have any of the meanings as given hereinbefore.

Particular compounds according to the invention indude those incorporated in Tables 1 and 2 and those specifically exemplified and named hereinafter including, without limitation:
7-(4-n-butylphenylsulfonyl)-1,2,3,5-tetrahydro-3-benzazepine; and
7-(4-n-butylphenylsulfonyl)-3-methyl-1,2,3,5-tetrahydro-3-benzazepine.

The compounds of the present invention may be in the form of their free base or physiologically acceptable salts thereof, particularly the monohydrochloride or monomesylate salts or pharmaceutically acceptable derivatives thereof.

The present invention also provides a general process (A) for preparing compounds of formula (I) which process comprises:
reacting a compound of formula (II) 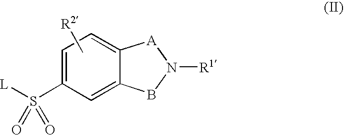
with a compound of formula (III) 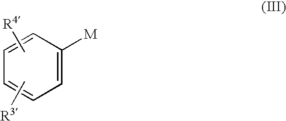
wherein L is a leaving group, such as fluoro, chloro, alkoxy or aryloxy, M is a metal, such as lithium or magnesium, R^{1′}-R^{4′} represent R¹ to R⁴ as hereinbefore defined or are groups that may be readily convertible to R¹ to R⁴, and A and B are as hereinbefore defined.

This general method (A) can be conveniently performed by mixing the two components at preferably −70° C. to room temperature in a suitable solvent such as tetrahydrofuran or ether for 10 minutes to 18 hours. Removal of certain R^{1,} protecting groups e.g. trifluoroacetyl, can also take place simultaneously during this process.

The present invention also provides a general process (B) for preparing compounds of formula (I), which process comprises:
reacting a compound of formula (IV) 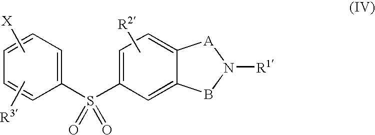
with an alkyl boronic acid of formula (V) 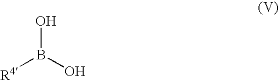
wherein X is a leaving group, such as bromo, iodo, chloro, triflate or N₂⁺. A and B are as hereinbefore defined and R^{1′}-R^{4′} represent R¹ to R⁴ as hereinbefore defined or are groups that may be readily convertible to R¹ to R⁴. This general method (B) can be conveniently performed by mixing the two components in a suitable solvent such as toluene or ethanol containing aqueous sodium carbonate and a catalytic amount of Pd(PPh₃)₄ at room temperature or reflux under argon.

The present invention also provides a general process (C) for preparing compounds of formula (I) which process comprises:
reacting a compound of formula (VI) 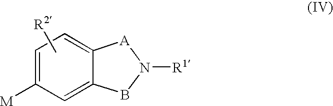
with a compound of formula (VII) 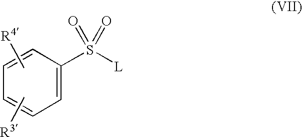
wherein L is a leaving group, such as fluoro, chloro, alkoxy or aryloxy, M is a metal, such as lithium or magnesium, A and B are as hereinbefore defined and R^{1′}-R^{4′} represent R¹ to R⁴ as hereinbefore defined or are groups that may be readily convertible to R¹ to R⁴. This general method (C) can be conveniently performed by mixing the two components at preferably −70° C. to room temperature in a suitable solvent such as tetrahydrofuran or ether for 10 minutes to 18 hours.

The present invention also provides a general process (D) for preparing compounds of formula (I) which process comprises:
reacting a reagent of formula (VIII) 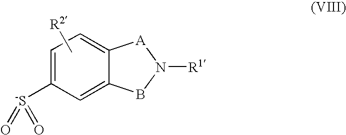
with a compound of formula (IX) 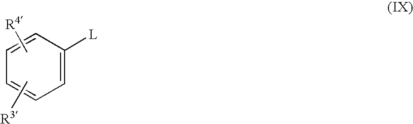
wherein L is a leaving group, such as fluoro, chloro or triflate, A and B are as hereinbefore defined and R^{1′}-R^{4′} represent R¹ to R⁴ as hereinbefore defined or are groups that may be readily convertible to R¹ to R⁴. This general method (D) can be conveniently performed by mixing the two components in a suitable solvent such as dimethylformamide in the presence of copper iodide at elevated temperature e.g. 120° C.

The present invention also provides a general process (E) for preparing compounds of formula (I) which process comprises:
reacting a reagent of formula (X) 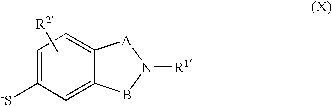
with a compound of formula (IX) 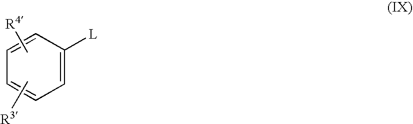
followed by the oxidation of the resultant sulfide, by for example, meta-chloroperbenzoic acid, wherein L is a leaving group, such as fluoro, chloro, triflate or N₂⁺, A and B are as hereinbefore defined and R^{1′}-R^{4′} represent R¹ to R⁴ as hereinbefore defined or are groups that may be readily convertible to R¹ to R⁴. This general method (E) can be conveniently performed by mixing the two components in a suitable solvent such as dimethylformamide, optionally at elevated temperature e.g. 120° C.

Interconversion of one of the R^{1′} to R^{5′} groups to the corresponding R¹ to R⁴ groups typically arises when one compound of formula (I) is used as the immediate precursor of another compound of formula (I), or when it is easier to introduce a more complex or reactive substituent at the end of a synthetic sequence.

For example, conversion of R^{1′} from a t-butoxycarbonyl (BOC) group to hydrogen is conducted by the treatment of the N-BOC protected compound with hydrogen chloride in ethanol or dioxan at room temperature.

Conversion of R^{1′} from hydrogen to an alkyl group is conducted by the treatment of the NH compound with the appropriate aldehyde in dichloroethane in the presence of a reducing agent, such as sodium triacetoxyborohydride, or by the treatment of the NH compound with the appropriate alkyl halide, such as iodomethane, under standard alkylation conditions (potassium carbonate in DMF at 60° C.).

Compounds of formula (II) are known in the literature or may be prepared by known processes, for example, chlorosulfonation of the aromatic ring using chlorosulfonic acid. Conversion to the suffonyl fluoride can be achieved, if required, by reaction with potassium fluoride in acetonitrile at room temperature. Suitable examples of an R^{1′} protecting group are trifluoroacetyl or the t-butoxycarbonyl (BOC) group.

Compounds of formula (III) are commercially available or may be prepared by established procedures, for example lithiation of the corresponding bromobenzene in tetrahydrofuran at low temperature, with for example t-butyl lithium.

Compounds of formula (IV) may be prepared using a similar process to general process A.

Compounds of formula (V) are commercially available, or may be prepared by lithiation of the corresponding bromo aromatic compound, followed by quenching with tri-isopropyl borate then hydrolysis.

Compounds of formula (VI) may be prepared by metal halogen exchange using the corresponding bromo analogue as starting material and t-butyl lithium at low temperature. Compounds of formula (VII) are commercially available or may be prepared by chlorosulfonylation of the aromatic ring. Conversion to the sulfonyl fluoride can be achieved, if required, by reaction with potassium fluoride in acetonitrile at room temperature.

Compounds of formula (VIII) may be prepared by reduction of the corresponding sulfonyl chloride, using for example sodium bisulphite and sodium bicarbonate in tetrahydrofuran/water. Deprotonation of the sulfinic add can be achieved by treatment with base, e.g. sodium hydride.

Compounds of formula (IX) are commercially available or may be prepared using standard literature methodology.

Compounds of formula (X) may be prepared by reduction of compounds of formula (II) using for example lithium aluminium hydride in tetrahydrofuran. Deprotonaton of the thiol can be achieved by treatment with base, e.g. sodium hydride.

Compounds of formula (I) have antagonist affinity for the serotonin 5-HT_{2C}, 5-HT_{2A} and 5-HT₆ receptors. These properties may give rise to anti-psychotic activity (e.g. improved effects on cognitive dysfunction) activity with reduced extrapyramidal side effects (eps), and/or anxiolytic/antidepressant activity. These could include, but are not limited to, attenuation of cognitive symptoms via 5-HT₆ receptor blockade (see Reavill, C. and Rogers, D. C., 2001, Investigational Drugs 2, 104-109), and reduced anxiety (see for example Kennett et al., Neuropharmacology 1997 April-May; 36 (4-5): 609-20), protection against EPS (Reavill et al., Brit. J. Pharmacol., 1999; 126: 572-574) and antidepressant activity (Bristow et al., Neuropharmacology 39:2000; 1222-1236) via 5-HT_{2C} receptor blockade.

Certain compounds of formula (I) have also been found to exhibit affinity for dopamine receptors, in particular the D₃ and D₂ receptors, and are useful in the treatment of disease states which require modulation of such receptors, such as psychotic conditions. Many of the compounds of formula (I) have also been found to have greater affinity for dopamine D₃ than for D₂ receptors. The therapeutic effect of currently available antipsychotic agents (neuroleptics) is generally believed to be exerted via blockade of D₂ receptors; however this mechanism is also thought to be responsible for undesirable eps associated with many neuroleptic agents. Without wishing to be bound by theory, it has been suggested that blockade of the dopamine D₃ receptor may give rise to beneficial antipsychotic activity without significant eps (see for example Sokoloff et al, Nature, 1990; 347: 146-151; and Schwartz et al, Clinical Neuropharmacology, Vol 16, No. 4, 295-314, 1993).

Compounds of formula (I) may also exhibit affinity for other receptors not mentioned above, resulting in beneficial antipyschotic activity.

The compounds of formula (I) are of use as antipsychotic agents for example in the treatment of schizophrenia, schizoaffective disorders, schizophreniform diseases, psychotic depression, mania, acute mania, paranoid and delusional disorders. Furthermore, they may have utility as adjunct therapy in Parkinsons Disease, particularly with compounds such as L-DOPA and possibly dopaminergic agonists, to reduce the side effects experienced with these treatments on long term use (e.g. see Schwartz et al., Brain Res. Reviews, 1998, 26, 236-242). From the localisation of D₃ receptors, it could also be envisaged that the compounds could also have utility for the treatment of substance abuse where it has been suggested that D3 receptors are involved (e.g. see Levant, 1997, Pharmacol. Rev., 49, 231-252). Examples of such substance abuse include alcohol, cocaine, heroin and nicotine abuse. Other conditions which may be treated by the compounds include dyskinetic disorders such as Parkinson's disease, neuroleptic-induced parkinsonism and tardive dyskinesias; depression; anxiety; agitation; tension; social or emotional withdrawal in psychotic patients; cognitive impairment including memory disorders such as Alzheimer's disease; psychotic states associated with neurodegenerative disorders, e.g. Alzheimer's disease; eating disorders; obesity; sexual dysfunction; sleep disorders; emesis; movement disorders; obsessive-compulsive disorders; amnesia; aggression; autism; vertigo; dementia; circadian rhythm disorders; and gastric motility disorders e.g. IBS.

Therefore, the invention provides a compound of formula (I) as hereinbefore described or a pharmaceutically acceptable salt or solvate thereof for use in therapy.

The invention also provides a compound of formula (I) or a pharmaceutically acceptable salt or solvate thereof for use in the treatment of a condition which requires modulation of a dopamine receptor.

The invention also provides a compound of formula (I) as hereinbefore described or a pharmaceutically acceptable salt or solvate thereof for use in the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides the use of a compound of formula (I) as hereinbefore described or a pharmaceutically acceptable salt or solvate thereof in the manufacture of a medicament for the treatment of a condition which requires modulation of a dopamine receptor.

The invention also provides the use of a compound of formula (I) as hereinbefore described or a pharmaceutically acceptable salt or solvate thereof in the manufacture of a medicament for the treatment of psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders.

The invention also provides a method of treating a condition which requires modulation of a dopamine receptor, which comprises administering to a mammal in need thereof an effective amount of a compound of formula (I) as hereinbefore described or a pharmaceutically acceptable salt or solvate thereof.

The invention also provides a method of treating psychotic disorders, schizophrenia, Parkinsons disease, substance abuse, dyskinetic disorders, depression, bipolar disorder, anxiety, cognitive impairment, eating disorders, obesity, sexual dysfunction, sleep disorders, emesis, movement disorders, obsessive-compulsive disorders, amnesia, aggression, autism, vertigo, dementia, circadian rhythm disorders and gastric motility disorders which comprises administering to a mammal in need thereof an effective amount of a compound of formula (I) as hereinbefore described or a pharmaceutically acceptable salt or solvate thereof.

A preferred use for dopamine antagonists according to the present invention is in the treatment of psychotic disorders, schizophrenia, Parkinsons disease, substance abuse, dyskinetic disorders, depression, bipolar disorder, anxiety and cognitive impairment. “Treatment” includes prophylaxis, where this is appropriate for the relevant condition(s).

It will be appreciated by those skilled in the art that the compounds according to the invention may advantageously be used in conjunction with one or more other therapeutic agents, for instance, different antidepressant agents such as 5HT₃ antagonists, serotonin agonists, NK-1 antagonists, selective serotonin reuptake inhibitors (SSRI), noradrenaline re-uptake inhibitors (SNRI), tricyclic antidepressants, dopaminergic antidepressants, H₃ antagonists, 5HT_{1A} antagonists, 5HT_{1B} antagonists, 5HT_{1D} antagonists, D₁ agonists, M₁ agonists and/or anticonvulsant agents.

Suitable 5HT₃ antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention indude imipramine, amitriptiline, chlomipranine and nortriptiline. Suitable dopaminergic antidepressants which may be used in combination with a compound of the invention indude bupropion and amineptine.

Suitable anticonvulsant agents which may be used in combination of the compounds of the inventions include for example divalproex, carbamazepine and diazepam.

It will be appreciated that the compounds of the combination or composition may be administered simultaneously (either in the same or different pharmaceutical formulations), separately or sequentially.

For use in medicine, the compounds of the present invention are usually administered as a standard pharmaceutical composition. The present invention therefore provides in a further aspect a pharmaceutical composition comprising a compound of formula (I) as hereinbefore described or a pharmaceutically (i.e. physiologically) acceptable salt thereof and a pharmaceutically (i.e. physiologically) acceptable carrier. The pharmaceutical composition can be for use in the treatment of any of the conditions described herein.

The compounds of formula (I) may be administered by any convenient method, for example by oral, parenteral (e.g. intravenous), buccal, sublingual, nasal, rectal or transdermal administration and the pharmaceutical compositions adapted accordingly.

The compounds of formula (I) as hereinbefore described and their pharmaceutically acceptable salts which are active when given orally can be formulated as liquids or solids, for example syrups, suspensions or emulsions, tablets, capsules and lozenges.

A liquid formulation will generally consist of a suspension or solution of the compound or pharmaceutically acceptable salt in a suitable liquid carrier(s) for example an aqueous solvent such as water, ethanol or glycerine, or a non-aqueous solvent, such as polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and cellulose.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the active substance in a pharmaceutically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a propellant which can be a compressed gas such as compressed air or an organic propellant such as a fluorochloro-hydrocarbon. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal administration are conveniently in the form of suppositories containing a conventional suppository base such as cocoa butter.

Compositions suitable for transdermal administration indude ointments, gels and patches. Preferably the composition is in unit dose form such as a tablet, capsule or ampoule.

Each dosage unit for oral administration contains preferably from 1 to 250 mg (and for parenteral administration contains preferably from 0.1 to 25 mg) of a compound of the formula (I) or a pharmaceutically acceptable salt thereof calculated as the free base.

The pharmaceutically acceptable compounds of the invention will normally be administered in a daily dosage regimen (for an adult patient) of, for example, an oral dose of between 1 mg and 500 mg, preferably between 10 mg and 400 mg, e.g. between 10 and 250 mg or an intravenous, subcutaneous, or intramuscular dose of between 0.1 mg and 100 mg, preferably between 0.1 mg and 50 mg, e.g. between 1 and 25 mg of the compound of the formula (I) or a pharmaceutically acceptable salt thereof calculated as the free base, the compound being administered 1 to 4 times per day. Suitably the compounds will be administered for a period of continuous therapy, for example for a week or more.

No toxicological effects are indicated/expected when a compound of the invention is administered in the above mentioned dosage range.

Biological Test Methods

Binding Experiments on Cloned Dopamine (e.g. D2 and D3) Receptors

The ability of the compounds to bind selectively to human D2/D3 dopamine receptors can be demonstrated by measuring their binding to cloned receptors. The inhibition constants (Kᵢ) of test compounds for displacement of [¹²⁵l]-lodosulpride binding to human D2/D3 receptors expressed in CHO cells were determined as follows. The cell lines were shown to be free from bacterial, fungal and mycoplasmal contaminants, and stocks of each were stored frozen in liquid nitrogen. Cultures were grown as monolayers or in suspension in standard cell culture media. Cells were recovered by scraping (from monolayers) or by centrifugation (from suspension cultures), and were washed two or three times by suspension in phosphate buffered saline followed by collection by centrifugation. Cell pellets were stored frozen at −80° C. Crude cell membranes were prepared by homogenisation followed by high-speed centrifugation, and characterisation of cloned receptors achieved by radioligand binding.

Preparation of CHO cell membranes: Cell pellets were gently thawed at room temperature, and resuspended in about 20 volumes of ice-cold Extraction buffer, 5 mM EDTA, 50 mM Trizma pre-set crystals (pH7.4@37° C.), 1 mM MgCl₂, 5 mM KCl and 120 mM NaCl. The suspension was homogenised using an Ultra-Turrax at full speed for 15 seconds. The homogenate was centrifuged at 18,000 r.p.m. for 15 min at 4° C. in a Sorvall RC5C centrifuge. Supernatant was discarded, and homogenate re-suspended in extraction buffer then centrifugation was repeated. The final pellet was resuspended in 50 mM Trizma pre-set crystals (pH 7.4@37° C.) and stored in 1 ml aliquot tubes at −80° C. (D2=3.0E+08 cells, D3=7.0E+07 cells and D4=1.0E+08 cells). The protein content was determined using a BCA protocol and bovine serum albumin as a standard (Smith, P. K., et al., Measurement of protein using bicinchoninic acid. Anal. Biochem. 150, 76-85 (1985)).

Binding experiments: Crude D2/D3 cell membranes were incubated with 0.03 nM [¹²⁵l]-lodosulpride (˜2000 Ci/mmol; Amersham, U. K., and the test compound in a buffer containing 50 mM Trizma pre-set crystals (pH 7.4@37° C.), 120 mM NaCl, 5 mM KCl, 2 mM CaCl₂, 1 mM MgCl₂, 0.3% (w/v) bovine serum albumin. The total volume is 0.2 ml and incubated in a water bath at 37° C. for 40 minutes. Following incubation, samples were filtered onto GF/B Unifilters using a Canberra Packard Filtermate, and washed four times with ice-cold 50 mM Trizma pre-set crystals (pH 7.4@37° C.). The radioactivity on the filters was measured using a Canberra Packard Topcount Scintillation counter. Non-specific binding was defined with 10 μM SKF-102161 (YM-09151). For competition curves, 10 serial log concentrations of competing cold drug were used (Dilution range: 10 μM-10 pM). Competition curves were analysed using Inflexion, an iterative curve fitting programme in Excel. Results were expressed as pKᵢ values where pKᵢ=−log10[Ki].

The exemplified compounds have pKᵢ values within the range of 5.8-8.2 at the dopamine D₃ receptor.

The exemplified compounds have pKᵢ values within the range of 5.3-7.7 at the dopamine D₂ receptor.

Binding Experiments on Cloned 5-HT₆ Receptors

Compounds can be tested following the procedures outlined in WO 98/27081.

The exemplified compounds have pKᵢ values within the range of 7.2-8.9 at the serotonin 5-HT₆ receptor.

Binding Experiments on Cloned 5-HT_{2A} and 5-HT_{2C} Receptors

Compounds can be tested following the procedures outlined in WO 94/04533.

The exemplified compounds have pKᵢ values within the range of 7.1-9.7 at the serotonin 5-HT_{2C} receptor and of 7.0-10.0 at the serotonin 5-HT_{2A} receptor.

The invention is further illustrated by the following non-limiting examples:

### Description 1

### 3-Trifluoroacetyl-1,2,4,5-tetrahydro-3-benzazepine-7-sulfonyl fluoride (D1)

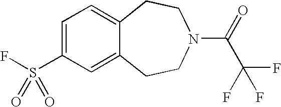

a) 3-Trifluoroacetyl-1,2,4,5-tetrahydro-3benzazepine-7-sulfonyl chloride

A solution of 3-trifluoroacetyl-1,2,4,5-tetrahydro-3-benzazepine (20 g, 80 mmol) in dichloromethane (50 ml) was added dropwise to a solution of chlorosulfonic acid (33 ml, 240 mmol) in more dichloromethane (200 ml) at 0° C. The resulting solution was stirred for 18 h without cooling then poured onto ice (250 g). The resulting organic layer was washed with brine (100 ml), dried (MgSO₄), and evaporated to give the subtitle compound as a white solid (23 g).

b) 3-Trifluoroacetyl-1,2,4,5tetrahydro-3-benzazepine-7-sulfonyl fluoride

A mixture of 3-trifluoroacetyl-1,2,4,5-tetrahydro-3-benzazepine-7-sulfonyl chloride (23 g, 67 mmol), potassium fluoride (12 g, 200mmol), 18-crown6 (0.1 g), and acetonitrile (100 ml) was stirred overnight. Water (200 ml) and ethyl acetate (200 ml) were added and the organic layer was washed with brine (100 ml), dried (MgSO₄), and evaporated to give the title compound as a white solid (21 g). ¹H NMR δ (d₆-DMSO) 3.2 (4H, m), 3.7 (4H, m), 7.6 (1H, m), and 8.0 (2H, m).

### Description 2

### 3-Trifluoroacetyl-8-methoxy-1,2,4,5-tetrahydro-3-benzazepine-7-sulfonyl fluoride (D2)

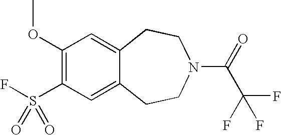

a) 3-Trifluoroacetyl-8-methoxy-1,2,4,5-tetrahydro3-benzazepine

To a mixture of 8-methoxy-1,2,4,5-tetrahydro-3-benzazepine hydrochloride (5.1 g, 25 mmol), triethylamine (8.4 ml, 60 mmol), and dichloromethane (100 ml) at 0° C., was added dropwise trifluoroacetic anhydride (3.5 ml, 26 mmol). The solution was stirred for 2 h without cooling then washed with saturated aqueous sodium hydrogen carbonate(100 ml), and water (100 ml). dried (MgSO₄), and evaporated to give the title compound as a white solid (5.5 g).

b) 3-Trifluoroacetyl-8-methoxy-1,2,4,5-tetrahydro-3-benzazepine-7-sulfonyl chloride

Prepared from 3-trifluoroacetyl-8-methoxy-1,2,4,5tetrahydro-3-benzazepine using the method of Description 1(a); yield 85%.

c) 3-Trifluoroacetyl-8-methoxy-1,2,4,5-tetrahydro-3-benzazepine-7-sulfonyl fluoride

Prepared from 3-trifluoroacetyl-8-methoxy-1,2,4,5-tetrahydro-3-benzazepine-7-sulfonyl chloride using the method of Description 1(b); yield 80%.

¹H NMR δ (d₆-DMSO) 3.1 (4H, m), 3.7 (4H, m), 4.0 (3H, s), 7.3 (1H, 2s, rotamers), and 7.8 (1H, 2s, rotamers).

### EXAMPLE 1

### 7-(4-n-Butylphenylsulfonyl)-1,2,4,5-tetrahydro-3-benzazepine (E1)

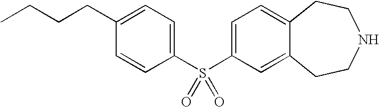

A solution of 4-n-butylbromobenzene (4.7 g, 22 mmol) in THF (35 ml) at −70° C. was treated with tert-butyllithium (25 ml, 1.7 M in pentane, 42 mmol). After 20 min at −70° C., a solution of D1 (2.7 g, 7.5 mmol in more THF (10 ml) was added, and after a further 30 min stirring without cooling, water (100 ml) and ethyl acetate (100 ml) were added. The organic layer was washed with brine (100 ml), dried (MgSO₄), and evaporated. Chromatography on silica, eluting with 0 to 15% methanol in dichloromethane containing 0.1 M ammonia, gave the title compound, isolated as the hydrochloride salt from ether (1.6 g). MH⁺ 344. ¹H NMR δ (d₆-DMSO) 0.9 (3H,t J=7 Hz), 1.3 (2H, m), 1.5 (2H, m), 2.6 (2H, t, J=8 Hz), 3.2 (8H, m), 7.4 (3H, m), 7.8 (4H, m), and 9.3 (2H, bs).

### EXAMPLE 2

### 7-(4-n-Butylphenylsulfonyl)-3-methyl-1,2,4,5-tetrahydro-3-benzazepine (E2)

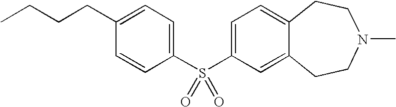

A mixture of E1 hydrochloride salt (1.6 g, 4.7 mmol), sodium triacetoxyborohydride (5.0 g), aqueous formaldehyde (5.0 ml, 37%), and 1,2-dichloroethane (100 ml) was stirred for 18 h then diluted with dichloromethane (50 ml) and washed with saturated aqueous sodium hydrogen carbonate (100 ml), dried (MgSO₄), and evaporated to give the title compound isolated as the hydrochloride salt from ether (1.3 g). MH⁺ 358. ¹H NMR δ (d₆-DMSO) 0.9 (3H, t J=7 Hz), 1.3 (2H, m), 1.5 (2H, m), 2.6 (2H, t, J=8 Hz), 2.8 (3H, d, J=5 Hz), 2.9-3.6 (8H, m), 7.4 (3H, m), 7.8 (4H, m), and 11.1 (1H, bs).

Examples 3-47 were prepared using analogous procedures to Examples 1 and 2. Products were isolated as either the free bases or hydrochloride salts. All ¹H NMR are consistent with the structures shown.

All of the compounds listed below in Table 1 relate to compounds of formula (I), (IA), (IB), (ID), (IE), (IJ) and (IL) wherein m and n both are 2:

TABLE 1

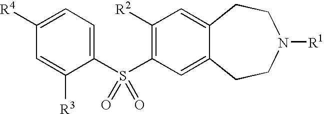

| Example | R¹ | R² | R³ | R⁴ | MH⁺ |
|---|---|---|---|---|---|
| 1 | H | H | H | nBu | 344 |
| 2 | Me | H | H | nBu | 358 |
| 3 | H | OMe | H | nBu | 374 |
| 4 | Me | OMe | H | nBu | 388 |
| 5 | H | H | H | tBu | 344 |
| 6 | Me | H | H | tBu | 358 |
| 7 | H | H | H | iPr | 330 |
| 8 | Me | H | H | iPr | 344 |
| 9 | Et | OMe | H | nBu | 402 |
| 10 | Me | OMe | H | iPr | 374 |
| 11 | H | OMe | H | iPr | 360 |
| 12 | H | OMe | H | tBu | 374 |
| 13 | Me | OMe | H | tBu | 388 |
| 14 | H | OH | H | nBu | 360 |
| 28 | H | H | H | OCF₃ | 372 |
| 29 | Me | H | H | OCF₃ | 386 |
| 30 | H | H | H | OnPr | 346 |
| 31 | H | H | H | OSO₂CF₃ | 436 |
| 32 | Me | H | H | OnPr | 360 |
| 33 | H | OMe | H | OCF₃ | 402 |
| 34 | Me | OMe | H | OCF₃ | 416 |
| 35 | H | OMe | H | OnPr | 376 |
| 36 | Me | OMe | H | OnPr | 390 |
| 48 | Me | OMe | H | OEt | 376 |
| 49 | Me | OMe | H | OCH₂CF₃ | 430 |
| 50 | Me | OMe | H | O-iPr | 390 |
| 51 | Me | OMe | H | OCH₂-tBu | 418 |
| 52 | Me | OMe | H | OCH₂-c-propyl | 402 |
| 53 | Me | OMe | H | O-c-pentyl | 416 |
| 54 | Me | OMe | Me | OnPr | 404 |

All of the compounds listed below in Table 2 relate to compounds of formula (I), (IA), (IB), (IC), (IJ) and (IK) wherein m and n both are 2:

TABLE 2

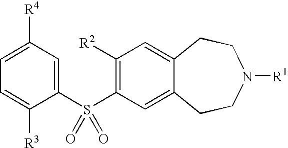

| Example | R¹ | R² | R³ | R⁴ | MH⁺ |
|---|---|---|---|---|---|
| 15 | H | H | H | nBu | 344 |
| 16 | Me | H | H | nBu | 358 |
| 17 | H | OMe | H | nBu | 374 |
| 18 | Me | OMe | H | nBu | 388 |
| 19 | iPr | OMe | H | nBu | 416 |
| 20 | H | H | H | iPr | 330 |
| 21 | Me | H | H | iPr | 344 |
| 22 | H | OMe | H | iPr | 360 |
| 23 | Me | OMe | H | iPr | 374 |
| 24 | H | H | OMe | nBu | 374 |
| 25 | Me | H | OMe | nBu | 388 |
| 26 | Me | H | OH | nBu | 374 |
| 27 | H | H | H | OH | 304 |
| 37 | H | H | H | OCF₃ | 372 |
| 38 | H | H | H | OiPr | 346 |
| 39 | Me | H | H | OCF₃ | 386 |
| 40 | Me | H | H | OiPr | 360 |
| 41 | H | H | H | OH | 304 |
| 42 | H | OMe | H | OCF₃ | 402 |
| 43 | H | OMe | H | OiPr | 376 |
| 44 | Me | OMe | H | OCF₃ | 416 |
| 45 | Me | OMe | H | OiPr | 390 |
| 46 | Me | H | H | OnPr | 360 |
| 47 | Me | OMe | H | OnPr | 390 |

All publications, including but not limited to patents and patent applications, cited in this specification are herein incorporated by reference as if each individual publication were specifically and individually indicated to be incorporated by reference herein as though fully set forth.

1. A compound of formula (I): 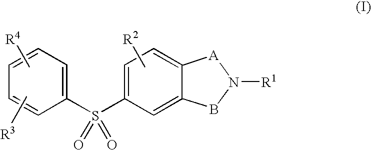
wherein
A and B represent the groups —(CH₂)ₘ— and —(CH₂)ₙ— respectively;
R¹ represents hydrogen or C₁₋₆alkyl;
R² represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁵R⁶, —SO₂NR⁵R⁶, —(CH₂)ₚNR⁵R⁶, —(CH₂)ₚNR⁵COR⁶,optionally substituted aryl ring, optionally substituted heteroaryl ring or optionally substituted heterocyclyl ring;
R³ represents hydrogen, halogen, hydroxy, cyano, nitro, hydroxyC₁₋₆alkyl, trifluoromethyl, trifluoromethoxy, C₁₋₆alkyl, C₁₋₆alkoxy, C₁₋₆fluoroalkoxy, —(CH₂)ₚC₃₋₆cycloalkyl, —(CH₂)ₚOC₃₋₆cycloalkyl, —COC₁₋₆alkyl, —SO₂C₁₋₆alkyl, —SOC₁₋₆alkyl, —S—C₁₋₆alkyl, —CO₂C₁₋₆alkyl, —CO₂NR⁷R⁸, —SO₂NR⁷R⁸, —(CH₂)ₚNR⁷R⁸ or —(CH₂)ₚNR⁷COR⁸;
R⁴ represents hydrogen, hydroxy, C₁₋₆alkyl, C₁₋₆alkoxy, C₁₋₆fluoroalkoxy, trifluoromethyl, trifluoromethoxy, halogen, —OSO₂CF₃, —(CH₂)ₚC₃₋₆cycloalkyl, —(CH₂)_{q}OC₁₋₆alkyl or —(CH₂)ₚOC₃₋₆cycloalkyl;
R⁵ and R⁶ each independently represent hydrogen, C₁₋₆alkyl or, together with the nitrogen or other atoms to which they are attached, form an azacycloalkyl ring or an oxo-substituted azacycloalkyl ring;
R⁷ and R⁸ each independently represent hydrogen or C₁₋₆alkyl;
m and n independently represent an integer selected from 1 and 2;
p independently represents an integer selected from 0, 1, 2 and 3;
q independently represents an integer selected from 1, 2 and 3;
or a pharmaceutically acceptable salt or solvate thereof,
with the proviso that the compounds 8-hydroxy-3-methyl-7-phenylsulfonyl-2,3,4,5-tetrahydro-1H-3-benzazepine, 8-hydroxy-7-(4-hydroxyphenyl)sulfonyl-2,3,4,5-tetrahydro-1H-3-benzazepine, 7-phenylsulfonyl-1,2,3,4-tetrahydroisoquinoline and 7-phenylsulfonyl-1,2,3,4-tetrahydroisoquinoline hydrochloride are excluded.

2. A compound of formula (I) according to claim 1 which is 7-(4-n-butylphenylsulfonyl)-1,2,3,5-tetrahydro-3-benzazepine; or
7-(4-n-butylphenylsulfonyl)-3-methyl-1,2,3,5-tetrahydro-3-benzazepine,
and pharmaceutically acceptable solvates thereof.

3. A pharmaceutical composition comprising a compound of formula (I) as claimed in claim 1 or a pharmaceutically acceptable salt thereof and a pharmaceutically acceptable carrier therefor.

4. A compound of formula (I) or a pharmaceutically acceptable salt or solvate thereof as claimed in claim 1 for use in therapy.

5. A method of treating a condition requiring modulation of a dopamine receptor comprising administering a compound of formula (I) or a pharmaceutically acceptable salt or solvate thereof as claimed in claim 1 to a mammal in need of such.

6. A method of treating psychotic disorders, Parkinsons disease, substance abuse, dyskinetic disorders, depression, bipolar disorder, anxity and cognitive impairment comprising administering a compound of formula (I) or a pharmaceutically acceptable salt or solvate thereof as claimed in claim 1.

7-8. (canceled)

9. A method of treating a condition which requires modulation of a dopamine receptor, which comprises administering to a mammal in need thereof an effective amount of a compound of formula (I) or a pharmaceutically acceptable salt or solvate thereof as claimed in claim 1.

10. A method of treating psychotic disorders, Parkinsons disease, substance abuse, dyskinetic disorders, depression, bipolar disorder, anxiety, and cognitive impairment which comprises administering to a mammal in need thereof an effective amount of a compound of formula (I) or a pharmaceutically acceptable salt or solvate thereof as claimed in claim 1.
